(19) United States
(12) Patent Application Publication
(10) Pub. No.: US 2023/0085830 A1
(43) Pub. Date: Mar. 23, 2023

(54) CATHETER AND TREATMENT METHOD

(71) Applicant: TERUMO KABUSHIKI KAISHA, Tokyo (JP)

(72) Inventors: Marcelo GUIMARAES, Mount Pleasant, SC (US); Ryo OKAMURA, Fujinomiya-shi (JP); Mitsuteru YASUNAGA, Fujinomiya-shi (JP)

(73) Assignee: TERUMO KABUSHIKI KAISHA, Tokyo (JP)

(21) Appl. No.: 18/060,173

(22) Filed: Nov. 30, 2022

Related U.S. Application Data
(63) Continuation of application No. PCT/JP2021/012976, filed on Mar. 26, 2021.

(30) Foreign Application Priority Data
Jun. 1, 2020 (JP) 2020-095742
Nov. 30, 2020 (JP) 2020-197870

(51) Int. Cl. A61M 25/00

(52) U.S. Cl. CPC A61M 25/0068

(57) ABSTRACT

A catheter is disclosed that can easily reach and be engaged with a uterine artery or a prostate artery from a radial artery and a treatment method. The catheter includes a tubular body having a lumen extending from a proximal end to a distal end. The tubular body includes, at a distal portion of the tubular body, a shaped portion shaped in a manner of being bent on substantially the same plane. The shaped portion includes a proximal bent portion bent to define a first angle, a distal bent portion defining a second angle distal of the proximal bent portion, an intermediate linear portion that is linear, and a distal linear portion that is linear. A first minimum curvature radius of the proximal bent portion is greater than 3 times and less than 11 times a second minimum curvature radius of the distal bent portion.

### CROSS-REFERENCES TO RELATED APPLICATIONS

This application is a continuation of International Application No. PCT/JP2021/012976 filed on Mar. 26, 2021, which claims priority to Japanese Patent Application No. 2020-095742 filed on Jun. 1, 2020, and Japanese Patent Application No. 2020-197870 filed on Nov. 30, 2020, the entire content of all three of which is incorporated herein by reference.

### TECHNOLOGICAL FIELD

The present disclosure generally relates to a catheter and a treatment method using a catheter.

### BACKGROUND DISCUSSION

Currently, intervention is performed in which treatment of a lesion area of a body is performed by an elongated catheter inserted into a blood vessel from a hole opened in a skin of a patient.

For example, U.S. Pat. No. 6,355,026 discloses percutaneous coronary intervention (PCI) of performing treatment of a coronary artery by trans radical intervention (TRI) of inserting a catheter from a radial artery of a wrist. Further, U.S. Pat. No. 7,422,581 discloses uterine fibroid embolization (UFE) of embolizing an uterine artery for treatment of a uterine fibroid by trans femoral intervention (TFI) of inserting a catheter from a femoral artery.

Inserting the catheter from the radial artery can reduce a physical burden on a patient, but the catheter that can be inserted is relatively thinner. In addition, in order for the catheter to reach the uterine artery or the prostate artery from the radial artery, it is necessary to move the catheter along a long path while crossing a plurality of branches of blood vessels. Therefore, for example, when a catheter for PCI is used, it is relatively difficult for the catheter to reach a target position.

### SUMMARY

A catheter is disclosed that can rather easily reach and be engaged with a uterine artery or a prostate artery from a radial artery, and a treatment method.

A catheter is disclosed for uterine artery engagement including a tubular body that includes a lumen extending from a proximal end of the tubular body to a distal end of the tubular body. The tubular body includes, at a distal portion of the tubular body, a shaped portion shaped in a manner of being bent on substantially the same plane, the shaped portion includes a proximal bent portion bent to define a first angle, a distal bent portion defining a second angle distal of the proximal bent portion and bent to the same side as the proximal bent portion, an intermediate linear portion that is linear and is disposed between the proximal bent portion and the distal bent portion, and a distal linear portion that is linear and is disposed distal of the distal bent portion, and a first minimum curvature radius which is a smallest curvature radius of an axis of the proximal bent portion is greater than 3 (three) times and less than 11 (eleven) times a second minimum curvature radius which is a smallest curvature radius of an axis of the distal bent portion.

In the catheter configured as described above, the proximal bent portion is in contact with the internal iliac artery, and the distal bent portion that is sharply bent to the same direction as the proximal bent portion with a curvature radius smaller than that of the proximal bent portion and/or the distal linear portion distal of the distal bent portion are easily directed toward the uterine artery and engaged with the uterine artery. Therefore, it is relatively easy for the catheter to reach the uterine artery from the radial artery and be engaged with the uterine artery.

A catheter is disclosed for prostate artery engagement including a tubular body communicating from a proximal end to a distal end. The tubular body includes, at a distal portion of the tubular body, a shaped portion shaped in a manner of being bent on substantially the same plane. The shaped portion includes a proximal bent portion defining a first angle, a distal bent portion defining a second angle distal of the proximal bent portion and bent to the same side as the proximal bent portion, an intermediate linear portion that is linear and is disposed between the proximal bent portion and the distal bent portion, and a distal linear portion that is linear and is disposed distal of the distal bent portion. A first minimum curvature radius which is a smallest curvature radius of an axis of the proximal bent portion is greater than 3 times and less than 11 times a second minimum curvature radius which is a smallest curvature radius of an axis of the distal bent portion.

In the catheter configured as described above, the proximal bent portion is in contact with the internal iliac artery, and the distal bent portion that is sharply bent to the same direction as the proximal bent portion with a curvature radius smaller than that of the proximal bent portion and/or the distal linear portion distal of the distal bent portion are easily directed toward the prostate artery and engaged with the prostate artery. Therefore, it is relatively easy for the catheter to reach the prostate artery from the radial artery and be engaged with the prostate artery.

The first minimum curvature radius may be 25 mm, the second minimum curvature radius may be 5 mm, the first minimum curvature radius may be 5 times the second minimum curvature radius, a length along an axis of the intermediate linear portion may be 16 mm, and a length along an axis of the distal linear portion may be 3 mm. Therefore, it is relatively easy for the catheter to reach and be engaged with the uterine artery or the prostate artery from the radial artery.

A catheter is disclosed comprising: a tubular body that includes a lumen extending from a proximal end of the tubular body to a distal end of the tubular body; the tubular body including, a shaped portion at a distal portion of the tubular body, the shaped portion being shaped in a manner of being bent on substantially the same plane; wherein the shaped portion includes: a proximal bent portion bent to define a first angle; a distal bent portion defining a second angle distal of the proximal bent portion and bent to the same side as the proximal bent portion; an intermediate linear portion that is linear and is disposed between the proximal bent portion and the distal bent portion; and a distal linear portion that is linear and is disposed distal of the distal bent portion; a first minimum curvature radius which is a smallest curvature radius of an axis of the proximal bent portion is greater than 3 times and less than 11 times a second minimum curvature radius which is a smallest curvature radius of an axis of the distal bent portion; wherein the first minimum curvature radius of the portion having the smallest curvature radius of the axis of the proximal bent portion is 15 mm to 50 mm, and the first angle is 15° to 70°; and wherein the second minimum curvature radius of the portion having the smallest curvature radius of the axis of the distal bent portion is 1 mm to 10 mm, and the second angle is 30° to 70°.

A treatment method is disclosed using a catheter including a tubular body communicating from a proximal end to a distal end, the treatment method includes: inserting, into a blood vessel from a radial artery, the catheter in which the tubular body includes, at a distal portion of the tubular body, a shaped portion shaped in a manner of being bent on substantially the same plane, the shaped portion includes a proximal bent portion defining a first angle, a distal bent portion defining a second angle distal of the proximal bent portion and bent to the same side as the proximal bent portion, an intermediate linear portion that is linear and is disposed between the proximal bent portion and the distal bent portion, and a distal linear portion that is linear and is disposed distal of the distal bent portion, and a first minimum curvature radius which is a smallest curvature radius of an axis of the proximal bent portion is greater than 3 times and less than 11 times a second minimum curvature radius which is a smallest curvature radius of an axis of the distal bent portion; and engaging the shaped portion with a blood vessel more peripheral than an internal iliac artery.

In the treatment method configured as described above, the proximal bent portion is brought into contact with the internal iliac artery, and the distal bent portion that is bent more sharply than the proximal bent portion in the same direction as the proximal bent portion and/or the distal linear portion distal of the distal bent portion are easily directed toward and engaged with the blood vessel more peripheral than the internal iliac artery. Therefore, it is easy for a surgeon to perform treatment in a favorable manner by causing the catheter to reach and be engaged with the blood vessel more peripheral than the internal iliac artery from the radial artery.

In the treatment method, the radial artery into which the catheter is to be inserted may be a distal radial artery. Accordingly, it is relatively easy for the surgeon to perform the treatment in a favorable manner by causing the catheter to reach and be engaged with the blood vessel more peripheral than the internal iliac artery in a less invasive manner than in a case of inserting the catheter from a conventional radial artery.

The blood vessel more peripheral than the internal iliac artery to be engaged with the shaped portion may be a prostate artery. Accordingly, the surgeon can treat the prostate in a favorable manner by using the catheter inserted into the blood vessel from the radial artery.

The blood vessel more peripheral than the internal iliac artery to be engaged with the shaped portion may be a uterine artery. Accordingly, the surgeon can treat a uterus in a favorable manner by using the catheter inserted into the blood vessel from the radial artery.

A treatment target may be an enlarged prostate due to the benign prostatic hyperplasia. Accordingly, the surgeon can treat the benign prostatic hyperplasia in a favorable manner by using the catheter inserted into the blood vessel from the radial artery.

A treatment target may be a uterine fibroid. Accordingly, the surgeon can treat the uterine fibroid in a favorable manner by using the catheter inserted into the blood vessel from the radial artery.

### BRIEF DESCRIPTION OF THE DRAWINGS

FIG. 1 is a schematic diagram showing an arrangement of a catheter in a blood vessel.

FIG. 2 is a plan view showing a distal portion of the catheter according to the embodiment.

FIG. 3 is a schematic diagram showing a state in which the catheter is inserted from a left radial artery, a proximal bent portion is attached to a left subclavian artery, a distal bent portion is disposed in an aorta, and a distal linear portion and a distal opening portion are directed to a descending aorta.

FIG. 4 is a schematic diagram showing a state in which the catheter is inserted into a common iliac artery and an internal iliac artery.

FIG. 5 is a schematic diagram showing a state in which the catheter is engaged with a uterine artery.

FIG. 6 is a schematic view showing another example of the state in which the catheter is engaged with the uterine artery.

FIG. 7 is a schematic diagram showing a state in which the catheter is engaged with a prostate artery.

### DETAILED DESCRIPTION

Set forth below with reference to the accompanying drawings is a detailed description of embodiments of a catheter and a treatment method using a catheter. Note that since embodiments described below are preferred specific examples of the present disclosure, although various technically preferable limitations are given, the scope of the present disclosure is not limited to the embodiments unless otherwise specified in the following descriptions. Note that for convenience of explanation, dimensions in the drawings may be exaggerated and may be different from actual dimensions. Further, in the present specification and the drawings, structural elements that have substantially the same function are denoted with the same reference numerals, and repeated explanation of these structural elements is omitted. In the present description, a side to be inserted into a blood vessel is referred to as a “distal side”, and a side to be operated is referred to as a “proximal side”.

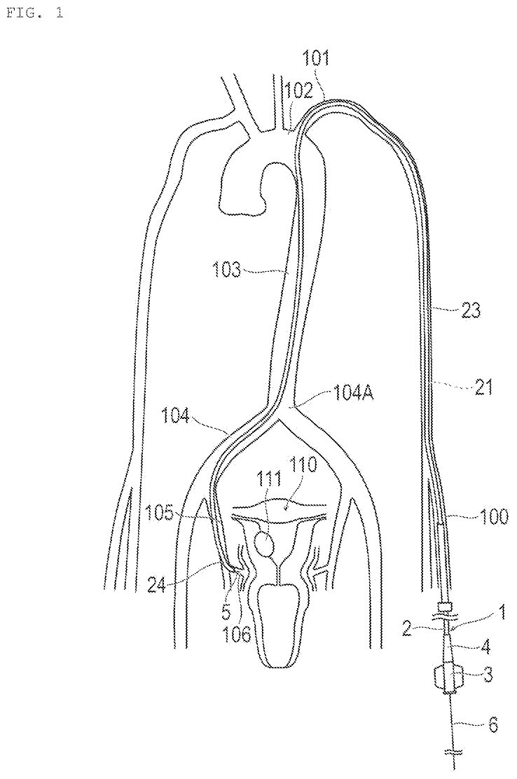

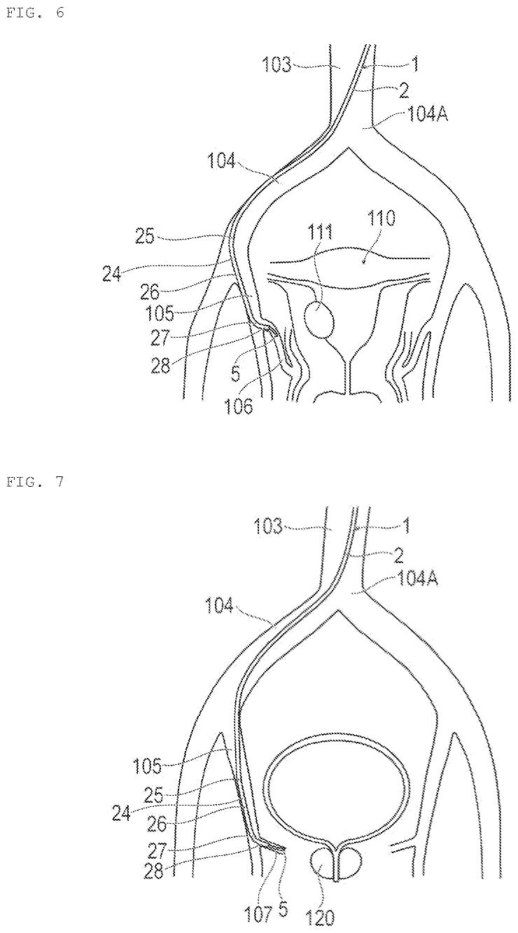

As shown in FIGS. 1 and 6, a catheter 1 according to the present embodiment is a catheter to be engaged with a uterine artery 106 or a prostate artery 107. The catheter 1 can be inserted into a blood vessel from a radial artery 100 of an arm of a patient and engaged with the uterine artery 106 or the prostate artery 107. The catheter 1 may be a so-called guiding catheter, an angiographic catheter, a guide wire support catheter, or a microcatheter. In addition, the catheter 1 may be a combination of a guiding catheter and a microcatheter that is inserted into an inner tubular of the guiding catheter and is longer than the guiding catheter.

As shown in FIG. 1, the catheter 1 includes a tubular body 2, a hub 3 disposed on a proximal side of the tubular body 2, and a strain relief device 4.

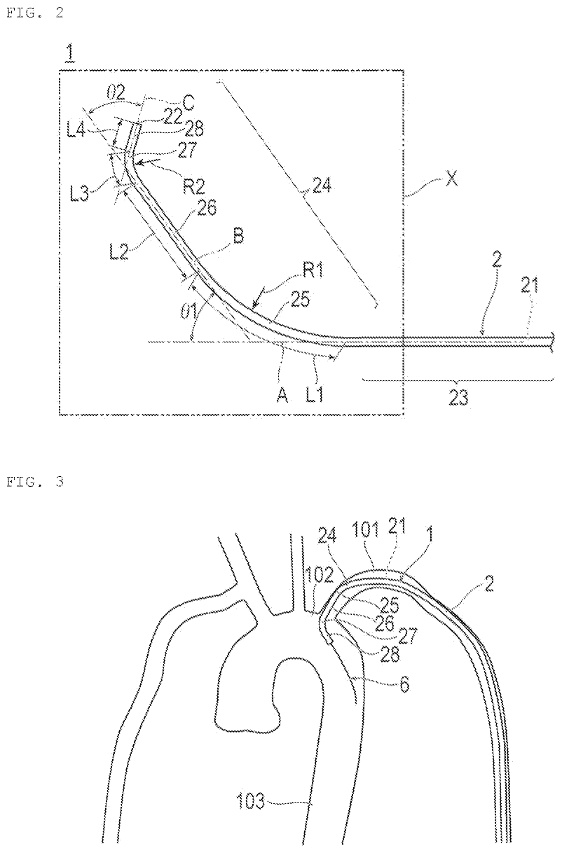

As shown in FIGS. 1 and 2, the tubular body 2 is elongated and flexible. The tubular body 2 is formed with a lumen 21 extending from a proximal end of the tubular body 2 to a distal end of the tubular body 2 in a substantially central portion (or middle portion) of the tubular body 2.

The hub 3 is formed with a passage communicating with the lumen 21. The hub 3 can be used to insert or remove, for example, a guide wire 6, a treatment catheter 5 (see FIG. 5), or the like. In addition, the hub 3 can be used to inject various liquids such as an X-ray contrast agent, a drug solution, and a saline solution (or saline).

The strain relief 4 is made of an elastic material. The strain relief device 4 covers a portion at which the tubular body 2 and the hub 3 are coupled to each other. Accordingly, the strain relief device 4 helps prevent the tubular body 2 from being bent or kinked in the vicinity of the portion at which the tubular body 2 and the hub 3 are coupled to each other.

Next, the tubular body 2 will be described in detail. The tubular body 2 includes a substantially linear main body portion 23 and a shaped portion 24 shaped in a bent state. An axis of the shaped portion 24 is located on the same plane X, and the shaped portion 24 is curved on substantially the same plane X. The axis means a line passing through a center of the tubular body 2, and means a central axis when the tubular body 2 is concentric circles. The shaped portion 24 includes a proximal bent portion 25, an intermediate linear portion 26, a distal bent portion 27, and a distal linear portion 28. The proximal bent portion 25 extends in a distal direction from a distal end of the main body portion 23 and is bent in the plane X. The intermediate linear portion 26 linearly extends in the distal direction from a distal end of the proximal bent portion 25. The distal bent portion 27 extends in the distal direction from a distal end of the intermediate linear portion 26 and is bent in the same direction as the proximal bent portion 25 in the plane X. The distal linear portion 28 linearly extends in the distal direction from a distal end of the distal bent portion 27. Note that the phrase “being curved on substantially the same plane X” may include not only being curved on the same plane X, but also a case in which a distal portion of the catheter 1 slightly protrudes from the plane X to such an extent that the same effect can be practically exhibited.

A line passing through the axis of the main body portion 23 is defined as a first axis A. A line passing through the axis of the intermediate linear portion 26 is defined as a second axis B. A line passing through the axis of the distal linear portion 28 is defined as a third axis C.

An angle formed by the second axis B on the distal side with respect to the first axis A is defined as a first angle 81. The first angle 81 is an amount of change in a direction of the axis of the tubular body 2 in the proximal bent portion 25. That is, the proximal bent portion 25 defines the first angle 81. When the first angle 81 is 0°, the second axis B coincides with the first axis A, and when the first angle 81 is 180°, the second axis B is folded back in an opposite direction with respect to the first axis A. A curvature radius of an axis of the proximal bent portion 25 may be constant throughout the entire proximal bent portion 25, or may vary depending on portions. In the proximal bent portion 25, a first minimum curvature radius R1 of a portion (a portion that is bent most sharply) having a smallest curvature radius of the axis is not particularly limited, and can be, for example, preferably 15 mm to 50 mm, and more preferably 25 mm. The first angle 81 is not particularly limited, and can be, for example, preferably 15° to 70°, and more preferably 55°.

An angle formed by the third axis C on the distal side with respect to the second axis B is defined as a second angle 82. The second angle 82 is an amount of change in the direction of the axis of the tubular body 2 in the distal bent portion 27. That is, the distal bent portion 27 defines the second angle 82. When the second angle 82 is 0°, the third axis C coincides with the second axis B, and when the second angle 82 is 180°, the third axis C is folded back in an opposite direction with respect to the second axis B. A curvature radius of an axis of the distal bent portion 27 may be constant throughout the entire distal bent portion 27, or may vary depending on portions. In the distal bent portion 27, a second minimum curvature radius R2 of a portion having a smallest curvature radius of the axis (a portion that is bent most sharply) is not particularly limited, and can be, for example, preferably 1 mm to 10 mm, and more preferably 5 mm. The second minimum curvature radius R2 is smaller than the first minimum curvature radius R1. The first minimum curvature radius R1 can be, for example, preferably 3 times or more and 11 times or less the second minimum curvature radius R2, and more preferably 5 times the second minimum curvature radius R2. The second angle 82 is not particularly limited, and can be, for example, preferably 30° to 70°, and more preferably 50°.

A length L1 along the axis of the proximal bent portion 25 is not particularly limited, and can be, for example, 1 mm to 100 mm. For example, the length L1 along the axis of the proximal bent portion 25 can be 24 mm.

A length L2 along the axis of the intermediate linear portion 26 is not particularly limited, and can be, for example, preferably 1 mm to 100 mm, and more preferably 16 mm.

A length L3 along the axis of the distal bent portion 27 is not particularly limited, and can be, for example, preferably 1 mm to 10 mm, and more preferably 4 mm.

A length L4 of the distal linear portion 28 is not particularly limited, and can be, for example, preferably 0.5 mm to 50 mm, and more preferably 3 mm.

In the tubular body 2, a reinforcement body may be embedded in a range from the proximal end to a predetermined position in the distal direction. The reinforcement body can be formed by braiding a plurality of wires into a tubular shape. A position of a most distal end of the reinforcement body is not particularly limited, and can be disposed in the shaped portion 24 or the main body portion 23. A flexible portion not provided with the reinforcement body can be provided distal of a reinforcement portion in which the reinforcement body is embedded. A length of the flexible portion can be, for example, preferably 500 mm or less, and more preferably 10 mm to 300 mm. For example, in a case of a catheter 1 of 4 Fr, the length of the flexible portion is 150 mm. In a case of a catheter 1 of 5 Fr, the length of the flexible portion is 150 mm.

The catheter 1 may be coated with a lubricating coating in a proximal direction along an axis from a distal end opening portion 22. The lubricating coating may be coated in the entire length of the catheter 1, and can be coated in, for example, a range of at least 500 mm or less, more preferably 400 mm or less in the proximal direction along the axis from the distal end opening portion 22. Therefore, a surgeon can deeply insert the catheter 1 into the uterine artery 106. Examples of the constituent material of the lubricating coating can include: a copolymer including an epoxy group-containing monomer such as glycidyl acrylate, glycidyl methacrylate, 3,4-epoxycyclohexylmethyl acrylate, 3,4-epoxycyclohexylmethyl methacrylate, β-methylglycidyl methacrylate, and allyl glycidyl ether, and a hydrophilic monomer such as N-methylacrylamide, N,N-dimethylacrylamide, and acrylamide; a (co)polymer including the hydrophilic monomer; a cellulose-based polymer material such as hydroxypropyl cellulose and carboxymethyl cellulose; a polysaccharide; a polyvinyl alcohol; a methyl vinyl ether-maleic anhydride copolymer; a water-soluble polyamide; poly(2-hydroxyethyl (meth)acrylate); polyethylene glycol; polyacrylamide; and polyvinyl pyrrolidone.

An outer diameter of the tubular body 2 can be, for example, preferably 1 mm (3 Fr) to 2.5 mm (6 Fr), and more preferably 1.3 mm (4 Fr) to 1.8 mm (5 Fr). An effective length of the tubular body 2 can be, for example, preferably 1300 mm to 1600 mm. It is preferable that a dimension of the tubular body 2 can be appropriately selected depending on the patient, a blood vessel to which the catheter 1 is to be introduced, or the like. For example, when the catheter 1 is introduced from the conventional radial artery 100 of a small-sized female, the effective length of the tubular body 2 is preferably relatively short. When the catheter 1 is introduced from a distal radial artery of a large-sized male, the effective length of the tubular body 2 is preferably relatively long. The effective length is a length of a portion that can be inserted into a blood vessel, a sheath, or the like. In the present embodiment, the effective length is a length from a most distal end of the strain relief 4 to a most distal end of the tubular body 2. In the present embodiment, the effective length of the catheter 1 for the uterine artery can be, for example, 1350 mm, the effective length of the catheter 1 for the prostate artery can be, for example, 1500 mm, and a length of the lubricating coating can be, for example, 400 mm from the distal end.

A distal tip that is more flexible than the proximal side may be disposed at the distal portion of the tubular body 2. The distal tip is made of a highly flexible material such as a rubber material.

The number of layers constituting the tubular body 2, a constituent material of each layer, presence or absence of the reinforcement body, and the like may be different depending on a position in a longitudinal direction of the tubular body 2.

Examples of the constituent material of the tubular body 2 can include various thermoplastic elastomers such as styrene-based, polyolefin-based, polyurethane-based, polyester-based, polyimide-based, polybutadiene-based, transpolyisoprene-based, fluororubber-based, and chlorinated polyethylene-based elastomers, polyetherketone, and polyimide-based ones, and a combination (polymer alloy, polymer blend, laminate, and the like) of one or two or more of above materials can be used. A low-friction material may be disposed on an inner peripheral surface of the tubular body 2 so that the guide wire 6 or another catheter may be easily inserted into the lumen 21. The low-friction material is, for example, a fluorine-based resin material such as polytetrafluoroethylene (PTFE). The tubular body 2 may contain a radiopaque material.

Next, a method of using the catheter 1 according to the present embodiment will be described.

As shown in FIG. 1, the catheter 1 is inserted into a blood vessel from the radial artery 100, passes through a brachial artery and a subclavian artery 101, and is inserted into a descending aorta 103. The catheter 1 is further inserted into a common iliac artery 104 through an aortic iliac artery bifurcated portion 104A of the common iliac artery 104, passes through an internal iliac artery 105, and is engaged with a blood vessel more peripheral than the internal iliac artery 105. The radial artery 100 to which the catheter 1 is to be introduced is, for example, a distal radial artery, a conventional radial artery, a sniff box radial artery, or the like. Here, a case in which the patient is a female and the catheter 1 is inserted into an artery of a left arm to treat a uterine fibroid 111 of a uterus 110 will be described as an example. Note that the catheter 1 may be inserted from the radial artery 100 of a right arm.

In a procedure, the surgeon inserts the guide wire 6 into the radial artery 100. Next, the surgeon pushes the catheter 1 in which the guide wire 6 is accommodated in the lumen 21 forward along the guide wire 6. Normally, a distal end of the guide wire 6 precedes the distal end of the catheter 1. Therefore, the shaped portion 24 of the catheter 1 is deformed by the guide wire 6 in the lumen 21 into a shape closer to a line than an original shape of the catheter 1. The shape closer to a straight than the original shape of the shaped portion 24 of the catheter 1 is not limited to a straight shape, and may be a bent shape.

As shown in FIG. 3, the guide wire 6 and the catheter 1 pass through the subclavian artery 101 and advance to an aortic arch 102. At a portion at which the subclavian artery 101 is connected to the aortic arch 102, the catheter 1 may need to be bent greatly in order to move toward the descending aorta 103. In order to cope with this need, for example, the surgeon can temporarily retract the guide wire 6 and accommodate the guide wire 6 in the lumen 21 of the catheter 1. A shape of a portion of the catheter 1 into which the guide wire 6 is not inserted is not limited by the guide wire 6. Therefore, a portion of the shaped portion 24 into which the guide wire 6 is not inserted attempts to restore an original bent shape. When the guide wire 6 retreats toward the proximal side from the shaped portion 24, the entire shaped portion 24 is restored to the original bent shape. Note that the distal end of the guide wire 6 may be positioned inside the shaped portion 24. In this state, the distal bent portion 27 is sharply bent to the same side as the proximal bent portion 25 with a curvature radius smaller than that of the proximal bent portion 25. Further, since the shaped portion 24 in the disclosure is smaller than a shaped portion of a catheter for coronary artery treatment, a most distal end of the catheter 1 can be rather easily directed to the descending aorta 103, not to an ascending aorta. The surgeon can advance the distal end of the catheter 1 from the aortic arch 102 toward the descending aorta 103 by using bending of the shaped portion 24. Thereafter, the surgeon causes the guide wire 6 to protrude from the catheter 1 toward the distal side. Accordingly, the guide wire 6 can rather easily advance to the descending aorta 103. Subsequently, the surgeon can push the catheter 1 forward along the guide wire 6. Accordingly, the catheter 1 can rather easily advance from the aortic arch 102 to the descending aorta 103. The catheter 1 moves to a peripheral side (a side close to a lower limb) in the descending aorta 103 and reaches the vicinity of the aortic iliac artery bifurcated portion 104A. Note that the surgeon may move the catheter 1 toward the peripheral side in the descending aorta 103 without causing the guide wire 6 to protrude from the catheter 1 toward the distal side.

After the catheter 1 reaches the vicinity of the aortic iliac artery bifurcated portion 104A, the surgeon retracts the guide wire 6 as necessary to accommodate the guide wire 6 in the lumen 21 of the catheter 1. Accordingly, the portion of the shaped portion 24 into which the guide wire 6 is not inserted is restored to the original bent shape. The surgeon can use the bending of the shaped portion 24 to direct the distal end of the catheter 1 toward a common iliac artery on a right or left side through the aortic iliac artery bifurcated portion 104A, thereby easily selecting the common iliac artery 104 on a desired side and inserting the distal end therein.

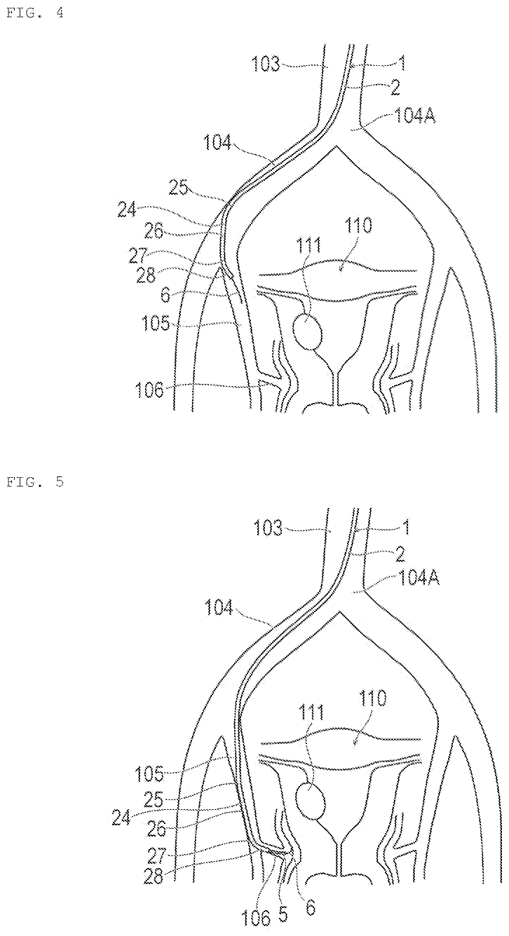

Thereafter, the surgeon causes the guide wire 6 to protrude from the catheter 1 toward the distal side. Accordingly, as shown in FIG. 4, the catheter 1 can rather easily advance through the common iliac artery 104. Note that the surgeon may move the catheter 1 toward the peripheral side in the common iliac artery 104 without causing the guide wire 6 to protrude from the catheter 1 toward the distal side. The catheter 1 moves through the common iliac artery 104 to the peripheral side and reaches the vicinity of an entrance of the internal iliac artery 105.

After the guide wire 6 and the catheter 1 reach the vicinity of the entrance of the internal iliac artery 105, the surgeon can retract the guide wire 6 as necessary to accommodate the guide wire 6 in the lumen 21 of the catheter 1. Accordingly, the portion of the shaped portion 24 into which the guide wire 6 is not inserted attempts to restore the original bent shape. In this state, the distal bent portion 27 is sharply bent to the same side as the proximal bent portion 25 with the curvature radius smaller than that of the proximal bent portion 25. Therefore, the most distal end of the catheter 1 is likely to be directed to the internal iliac artery 105 bifurcating from the common iliac artery 104. The surgeon can use the bending of the shaped portion 24 to direct the distal end of the catheter 1 to the entrance of the internal iliac artery 105 and rather easily insert the distal end without damaging the internal iliac artery 105, which has an inner diameter smaller than that of the common iliac artery 104.

Thereafter, the surgeon causes the guide wire 6 to protrude from the catheter 1 toward the distal side. Accordingly, the catheter 1 can rather easily advance through the internal iliac artery 105. Note that the surgeon may move the catheter 1 toward a peripheral side in the internal iliac artery 105 without causing the guide wire 6 to protrude from the catheter 1 toward the distal side. The catheter 1 moves through the internal iliac artery 105 to the peripheral side and reaches the vicinity of an entrance of the uterine artery 106.

After the guide wire 6 and the catheter 1 reach the vicinity of the entrance of the uterine artery 106, the surgeon retracts the guide wire 6 as necessary to accommodate the guide wire 6 in the lumen 21 of the catheter 1. Accordingly, as shown in FIG. 5, the portion of the shaped portion 24 into which the guide wire 6 is not inserted attempts to restore the original bent shape. The surgeon can use the bending of the shaped portion 24 to direct the distal end of the catheter 1 to the entrance of the uterine artery 106, and to insert the distal end of the catheter 1 rather easily into the uterine artery 106. In this state, the distal bent portion 27 is sharply bent to the same side as the proximal bent portion 25 with the curvature radius smaller than that of the proximal bent portion 25. Therefore, the most distal end of the catheter 1 is more likely to be directed to the uterine artery 106 bifurcating from the internal iliac artery 105, which is thinner than the internal iliac artery 105. Therefore, the distal bent portion 27 and/or the distal linear portion 28 of the catheter 1 are in contact with a blood vessel wall of the uterine artery 106, and the proximal bent portion 25 is in contact with and can be stably held by a blood vessel wall on a side opposite to an entrance portion of the uterine artery 106 of a blood vessel wall of the internal iliac artery 105. Accordingly, the distal bent portion 27 and/or the distal linear portion 28 of the catheter 1 can be engaged with the uterine artery 106 in a favorable manner, and positions of the distal bent portion 27 and/or the distal linear portion 28 of the catheter can be easily maintained. The distal bent portion 27 and/or the distal linear portion 28 may or may not be in contact with the blood vessel wall of the uterine artery 106.

Thereafter, the treatment catheter 5, which is longer than the catheter 1, is inserted into the lumen 21 of the catheter 1 from the hub 3. The surgeon can rather easily insert the treatment catheter 5 into the uterine artery 106 by protruding the treatment catheter 5 from the distal end of the catheter 1. Thereafter, the surgeon can release an embolic agent and/or a drug solution for occluding the uterine fibroid 111 of the uterus 110 through the treatment catheter 5. The catheter 1 relaxes a reaction generated by releasing the embolic agent, the drug solution, or the like from the treatment catheter 5. Further, the catheter 1 can apply a backup force to the treatment catheter 5 to hold the treatment catheter 5 at a desired position. Therefore, the distal end of the catheter 1 can be prevented from coming out of the entrance of the uterine artery 106 by a reaction generated when the treatment catheter 5 such as a microcatheter is inserted into the lumen 21 of the catheter 1 or when the embolic agent, the drug solution, or the like is released. Note that a medical device to be inserted into the catheter 1 may not be the treatment catheter 5. The surgeon may release the embolic agent and/or the drug solution from the catheter 1 instead of the treatment catheter 5.

Alternatively, as shown in FIG. 6, when the internal iliac artery 105 is relatively short, the surgeon can use the bending of the shaped portion 24 to direct the distal end of the catheter 1 toward the entrance of the uterine artery 106, attach the distal bent portion 27 to the internal iliac artery 105, and attach the proximal bent portion 25 to the common iliac artery 104. In this state, the distal end of the catheter 1 can be prevented from coming out of the entrance of the uterine artery 106 by the reaction generated when the treatment catheter 5 such as a microcatheter is inserted into the lumen 21 of the catheter 1 or when the embolic agent, the drug solution, or the like is released.

Alternatively, the distal bent portion 27 may be engaged with a uterine artery ascending branch, the embolic agent may be injected toward a peripheral artery, and the surgeon may release the embolic agent and/or the drug solution by using the catheter 1 and the treatment catheter 5, or may release the embolic agent and/or the drug solution by using the catheter 1 alone.

Note that the catheter 1 may be used to treat benign prostatic hyperplasia of a prostate 120 of a male patient. In this case, an operation of the catheter 1 until the catheter 1 is inserted into the internal iliac artery 105 is the same as the operation of the catheter 1 in the treatment of the uterine fibroid 111. The guide wire 6 and the catheter 1 pass through the internal iliac artery 105 and reach the vicinity of an entrance of the prostate artery 107. The prostate artery 107 is an artery that nourishes the prostate 120, and can be, for example, an inferior vesical artery, a middle rectal artery, an internal pudendal artery, or an obturator artery. After the guide wire 6 and the catheter 1 reach the vicinity of the entrance of the prostate artery 107, the surgeon retracts the guide wire 6 as necessary to accommodate the guide wire 6 in the lumen 21 of the catheter 1. Accordingly, as shown in FIG. 7, the portion of the shaped portion 24 into which the guide wire 6 is not inserted attempts to restore the original bent shape. The surgeon can use the bending of the shaped portion 24 to direct the distal end of the catheter 1 to the entrance of the prostate artery 107, and can rather easily insert the distal end into the prostate artery 107. In this state, the distal bent portion 27 is bent to the same side as the proximal bent portion 25. Therefore, the most distal end of the catheter 1 is more likely to be directed to the prostate artery 107 bifurcating from the internal iliac artery 105, which is thinner than the internal iliac artery 105. Therefore, the distal bent portion 27 and/or the distal linear portion 28 of the catheter 1 are in contact with a blood vessel wall of the prostate artery 107, and the proximal bent portion 25 is in contact with and stably held by a blood vessel wall on a side opposite to an entrance portion of the prostate artery 107 of a blood vessel wall of the internal iliac artery 105. Accordingly, the distal bent portion 27 and/or the distal linear portion 28 of the catheter 1 can be engaged with the prostate artery 107 in a favorable manner, and positions of the distal bent portion 27 and/or the distal linear portion 28 of the catheter 1 can be rather easily maintained.

Thereafter, the treatment catheter 5, which is longer than the catheter 1, is inserted into the lumen 21 of the catheter 1 from the hub 3. The surgeon can rather easily insert the treatment catheter 5 into the prostate artery 107 by protruding the treatment catheter 5 from the distal end of the catheter 1. Thereafter, the surgeon can release an embolic agent and/or a drug solution that reduces a blood flow to the prostate via the treatment catheter 5. According to the catheter 1 of the present embodiment, the distal end of the catheter 1 can be prevented from coming out of the entrance of the prostate artery 107 by a reaction generated when the treatment catheter 5 such as a microcatheter is inserted into the lumen 21 of the catheter 1 or when the embolic agent, the drug solution, or the like is released. Note that when the internal iliac artery 105 is relatively short, the surgeon can use the bending of the shaped portion 24 to direct the distal end of the catheter 1 toward the entrance of the prostate artery 107, attach the distal bent portion 27 to the internal iliac artery 105, and attach the proximal bent portion 25 to the common iliac artery 104. Note that a medical device to be inserted into the catheter 1 may not be the treatment catheter 5. The surgeon may release the embolic agent and/or the drug solution from the catheter 1 instead of the treatment catheter 5.

As described above, the catheter 1 according to the present embodiment is the catheter 1 for uterine artery engagement including a tubular body communicating from a proximal end to a distal end. The tubular body 2 can include, at a distal portion of the tubular body 2, the shaped portion 24 shaped in a manner of being bent on substantially the same plane X. The shaped portion 24 includes the proximal bent portion 25 bent to define the first angle 81, the distal bent portion 27 defining the second angle 82 distal of the proximal bent portion 25 and bent to the same side as the proximal bent portion 25, the intermediate linear portion 26 that is linear and is disposed between the proximal bent portion 25 and the distal bent portion 27, and the distal linear portion 28 that is linear and is disposed distal of the distal bent portion 27. The first minimum curvature radius R1 which is a smallest curvature radius of an axis of the proximal bent portion 25 can be, for example, greater than 3 times and less than 11 times a second minimum curvature radius R2 which is a smallest curvature radius of an axis of the distal bent portion 27.

In the catheter 1 configured as described above, the proximal bent portion 25 is in contact with the internal iliac artery 105, and the distal bent portion 27 that is sharply bent to the same direction as the proximal bent portion 25 with a curvature radius smaller than that of the proximal bent portion 25 and/or the distal linear portion 28 disposed distal of the distal bent portion 27 are rather easily directed toward the uterine artery 106 and engaged with the uterine artery 106. Therefore, it can be rather easy for the catheter 1 to reach the uterine artery 106 from the radial artery 100 and be engaged with the uterine artery 106.

Further, the catheter 1 according to the present embodiment is the catheter 1 for prostate artery engagement including the tubular body 2 communicating from a proximal end to a distal end. The tubular body 2 can include, at a distal portion of the tubular body 2, the shaped portion 24 shaped in a manner of being bent on substantially the same plane X. The shaped portion 24 can include the proximal bent portion 25 bent so as to define the first angle 81, the distal bent portion 27 defining the second angle 82 distal of the proximal bent portion 25 and bent to the same side as the proximal bent portion 25, the intermediate linear portion 26 that is linear and is disposed between the proximal bent portion 25 and the distal bent portion 27, and the distal linear portion 28 that is linear and is disposed distal of the distal bent portion 27. The first minimum curvature radius R1 which is a smallest curvature radius of an axis of the proximal bent portion 25 can be, for example, greater than 3 times and less than 11 times the second minimum curvature radius R2 which is a smallest curvature radius of an axis of the distal bent portion 27.

In the catheter 1 configured as described above, the proximal bent portion 25 is in contact with the internal iliac artery 105, and the distal bent portion 27 that is sharply bent to the same direction as the proximal bent portion 25 with a curvature radius smaller than that of the proximal bent portion 25 and/or the distal linear portion 28 disposed distal of the distal bent portion 27 are rather easily directed toward the prostate artery 107 and engaged with the prostate artery 107. Therefore, it can be rather easy for the catheter 1 to reach the prostate artery 107 from the radial artery and be engaged with the prostate artery 107.

Further, for example, the first minimum curvature radius R1 may be 25 mm, the second minimum curvature radius R2 may be 5 mm, the first minimum curvature radius R1 which is the smallest curvature radius of the axis of the proximal bent portion 25 may be 5 times the second minimum curvature radius R2 which is the smallest curvature radius of the axis of the distal bent portion, a length along the axis of the intermediate linear portion 26 may be 16 mm, and a length along the axis of the distal linear portion 28 may be 3 mm. Therefore, it is rather easy for the catheter 1 to reach the uterine artery 106 or the prostate artery 107 from the radial artery 100 and be engaged with the uterine artery 106 or the prostate artery 107.

The disclosure further provides a treatment method using the catheter 1 including the tubular body 2 communicating from the proximal end to the distal end. The present treatment method can include: inserting, into a blood vessel from a radial artery, the catheter 1 in which the tubular body 2 includes, at the distal portion of the tubular body 2, the shaped portion 24 shaped in a manner of being bent on substantially the same plane X, the shaped portion 24 includes the proximal bent portion 25 bent to define the first angle 81, the distal bent portion 27 defining the second angle 82 distal of the proximal bent portion 25 and bent to the same side as the proximal bent portion 25, the intermediate linear portion 26 that is linear and is disposed between the proximal bent portion 25 and the distal bent portion 27, and the distal linear portion 28 that is linear and is disposed distal of the distal bent portion 27, and the minimum curvature radius R1 of the proximal bent portion 25 is greater than 3 times and less than 11 times the minimum curvature radius R2 of the distal bent portion 27; and engaging the shaped portion 24 with a blood vessel more peripheral than the internal iliac artery 105.

In the treatment method configured as described above, the proximal bent portion 25 is brought into contact with the internal iliac artery 105 to be stably held by the blood vessel wall, and the distal bent portion 27 that is sharply bent to the same direction as the proximal bent portion 25 with the curvature radius smaller than that of the proximal bent portion 25 and/or the distal linear portion 28 disposed distal of the distal bent portion 27 are rather easily engaged with the blood vessel more peripheral than the internal iliac artery 105. Therefore, it is rather easy for the surgeon to perform the treatment in a favorable manner by causing the catheter 1 to reach and be engaged with the blood vessel more peripheral than the internal iliac artery 105 from the radial artery 100.

In the treatment method, the radial artery 100 into which the catheter 1 is to be inserted may be the distal radial artery. Accordingly, it is relatively easy for the surgeon to perform the treatment in a favorable manner by causing the catheter 1 to reach and be engaged with the blood vessel more peripheral than the internal iliac artery 105 in a less invasive manner than in a case of inserting the catheter 1 from the conventional radial artery.

The blood vessel that is disposed on the peripheral side relative to the internal iliac artery 105 to be engaged with the shaped portion 24 may be the uterine artery 106. Accordingly, the surgeon can treat the uterus 110 in a favorable manner by using the catheter 1 inserted into the blood vessel from the radial artery 100.

A treatment target may be the uterine fibroid 111. Accordingly, the surgeon can treat the uterine fibroid 111 in a favorable manner by using the catheter 1 inserted into the blood vessel from the radial artery 100.

The blood vessel that is disposed on the peripheral side relative to the internal iliac artery 105 to be engaged with the shaped portion 24 may be the prostate artery 107. Accordingly, the surgeon can treat the prostate 120 (for example, treat the benign prostatic hyperplasia) in a favorable manner by using the catheter 1 inserted into the blood vessel from the radial artery 100.

A treatment target can be, for example, the enlarged prostate 120 due to the benign prostatic hyperplasia. Accordingly, the surgeon can treat the benign prostatic hyperplasia in a favorable manner by using the catheter 1 inserted into the blood vessel from the radial artery 100.

The detailed description above describes embodiments of a catheter and a treatment method using a catheter. These disclosed embodiments represent examples of the catheter and the treatment method using the catheter. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A catheter comprising:
a tubular body including a lumen extending from a proximal end of the tubular body to a distal end of the tubular body;
a shaped portion at a distal portion of the tubular body, the shaped portion being shaped in a manner of being bent on substantially the same plane;
wherein the shaped portion includes:
a proximal bent portion bent to define a first angle;
a distal bent portion defining a second angle distal of the proximal bent portion and bent to the same side as the proximal bent portion;
an intermediate linear portion that is linear and is disposed between the proximal bent portion and the distal bent portion; and
a distal linear portion that is linear and is disposed distal of the distal bent portion; and
a first minimum curvature radius which is a smallest curvature radius of an axis of the proximal bent portion is greater than 3 times and less than 11 times a second minimum curvature radius which is a smallest curvature radius of an axis of the distal bent portion.

2. The catheter according to claim 1, wherein the first minimum curvature radius is 25 mm, the second minimum curvature radius is 5 mm, the first minimum curvature radius is 5 times the second minimum curvature radius, a length along an axis of the intermediate linear portion is 16 mm, and a length along an axis of the distal linear portion is 3 mm.

3. The catheter according to claim 1, wherein the catheter is a guiding catheter, an angiographic catheter, a guide wire support catheter, or a microcatheter.

4. The catheter according to claim 3, wherein
the proximal bent portion extends in a distal direction from a distal end of a main body portion of the tubular body;
the intermediate linear portion extends in the distal direction from a distal end of the proximal bent portion;
the distal bent portion extends in the distal direction from a distal end of the intermediate linear portion and is bent in a same direction as the proximal bent portion; and
the distal linear portion extends in the distal direction from a distal end of the distal bent portion.

5. The catheter according to claim 1,
wherein a line passing through an axis of the main body portion is defined as a first axis, a line passing through an axis of the intermediate linear portion is defined as a second axis, a line passing through an axis of the distal linear portion is defined as a third axis; and
wherein an angle formed by the second axis on the distal side with respect to the first axis is defined as the first angle, the first angle is an amount of change in a direction of the axis of the tubular body in the proximal bent portion, the proximal bent portion defines the first angle, and when the first angle is 0°, the second axis coincides with the first axis, and when the first angle is 180°, the second axis is folded back in an opposite direction with respect to the first axis.

6. The catheter according to claim 1, wherein the first minimum curvature radius of the portion having the smallest curvature radius of the axis of the proximal bent portion is 15 mm to 50 mm, and the first angle is 15° to 70°.

7. The catheter according to claim 1, wherein an angle formed by the third axis on the distal side with respect to the second axis is defined as the second angle, the second angle is an amount of change in the direction of the axis of the tubular body in the distal bent portion, and when the second angle is 0°, the third axis coincides with the second axis, and when the second angle is 180°, the third axis is folded back in an opposite direction with respect to the second axis.

8. The catheter according to claim 1, wherein the second minimum curvature radius of the portion having the smallest curvature radius of the axis of the distal bent portion is 1 mm to 10 mm, and the second angle is 30° to 70°.

9. The catheter according to claim 1, wherein the first minimum curvature radius is 5 times the second minimum curvature radius.

10. A catheter comprising:
a tubular body that includes a lumen extending from a proximal end of the tubular body to a distal end of the tubular body;
the tubular body including, a shaped portion at a distal portion of the tubular body, the shaped portion being shaped in a manner of being bent on substantially the same plane;
wherein the shaped portion includes:
a proximal bent portion bent to define a first angle;
a distal bent portion defining a second angle distal of the proximal bent portion and bent to the same side as the proximal bent portion;
an intermediate linear portion that is linear and is disposed between the proximal bent portion and the distal bent portion; and
a distal linear portion that is linear and is disposed distal of the distal bent portion;
a first minimum curvature radius which is a smallest curvature radius of an axis of the proximal bent portion is greater than 3 times and less than 11 times a second minimum curvature radius which is a smallest curvature radius of an axis of the distal bent portion;
wherein the first minimum curvature radius of the portion having the smallest curvature radius of the axis of the proximal bent portion is 15 mm to 50 mm, and the first angle is 15° to 70°; and
wherein the second minimum curvature radius of the portion having the smallest curvature radius of the axis of the distal bent portion is 1 mm to 10 mm, and the second angle is 30° to 70°.

11. The catheter according to claim 10, wherein the first minimum curvature radius is 25 mm, the second minimum curvature radius is 5 mm, the first minimum curvature radius is 5 times the second minimum curvature radius, a length along an axis of the intermediate linear portion is 16 mm, and a length along an axis of the distal linear portion is 3 mm.

12. The catheter according to claim 10, wherein
the proximal bent portion extends in a distal direction from a distal end of a main body portion of the tubular body;
the intermediate linear portion extends in the distal direction from a distal end of the proximal bent portion;
the distal bent portion extends in the distal direction from a distal end of the intermediate linear portion and is bent in a same direction as the proximal bent portion; and
the distal linear portion extends in the distal direction from a distal end of the distal bent portion.

13. The catheter according to claim 10,
wherein a line passing through an axis of the main body portion is defined as a first axis, a line passing through an axis of the intermediate linear portion is defined as a second axis, a line passing through an axis of the distal linear portion is defined as a third axis; and
wherein an angle formed by the second axis on the distal side with respect to the first axis is defined as the first angle, the first angle is an amount of change in a direction of the axis of the tubular body in the proximal bent portion, the proximal bent portion defines the first angle, and when the first angle is 0°, the second axis coincides with the first axis, and when the first angle is 180°, the second axis is folded back in an opposite direction with respect to the first axis.

14. The catheter according to claim 10, wherein an angle formed by the third axis on the distal side with respect to the second axis is defined as the second angle, the second angle is an amount of change in the direction of the axis of the tubular body in the distal bent portion, and when the second angle is 0°, the third axis coincides with the second axis, and when the second angle is 180°, the third axis is folded back in an opposite direction with respect to the second axis.

15. A treatment method using a catheter, the catheter including a tubular body that includes a lumen extending from a proximal end of the tubular body to a distal end of the tubular body, the treatment method comprising:
inserting, into a blood vessel from a radial artery, the catheter in which the tubular body includes a shaped portion on a distal end of the tubular body, the shaped portion being shaped in a manner of being bent on substantially the same plane, the shaped portion includes a proximal bent portion bent to define a first angle, a distal bent portion defining a second angle distal of the proximal bent portion and bent to the same side as the proximal bent portion, an intermediate linear portion that is linear and is disposed between the proximal bent portion and the distal bent portion, and a distal linear portion that is linear and is disposed distal of the distal bent portion, and a first minimum curvature radius which is a smallest curvature radius of an axis of the proximal bent portion is greater than 3 times and less than 11 times a second minimum curvature radius which is a smallest curvature radius of an axis of the distal bent portion; and
engaging the shaped portion with a blood vessel more peripheral than an internal iliac artery.

16. The treatment method according to claim 15,
wherein the radial artery into which the catheter is to be inserted is a distal radial artery.

17. The treatment method according to claim 15,
wherein the blood vessel more peripheral than the internal iliac artery to be engaged with the shaped portion is a prostate artery.

18. The treatment method according to claim 15,
wherein the blood vessel more peripheral than the internal iliac artery to be engaged with the shaped portion is a uterine artery.

19. The treatment method according to claim 17,
wherein a treatment target is an enlarged prostate due to benign prostatic hyperplasia.

20. The treatment method according to claim 18,
wherein a treatment target is a uterine fibroid.
